SEQUENCE LISTING (18 sequences)

SEQ ID NO: 1 - DNA, 24 bp, synthetic construct
  source 1..24: mol_type = other DNA
ccgtggggtattctcctttatcta

SEQ ID NO: 2 - DNA, 22 bp, synthetic construct
  source 1..22: mol_type = other DNA
ctccgtggggtattctccttta

SEQ ID NO: 3 - DNA, 27 bp, synthetic construct
  source 1..27: mol_type = other DNA
gagatctccaaactagtctctacagaa

SEQ ID NO: 4 - DNA, 20 bp, synthetic construct
  source 1..20: mol_type = other DNA
tgacgcctgagatctccaaa

SEQ ID NO: 5 - DNA, 29 bp, synthetic construct
  source 1..29: mol_type = other DNA
  misc_feature 1: note = quencher attached to a nucleotide sequence
  misc_feature 29: note = nucleotide attached to a blocker
cctgctccaacagttagtgatgctcggaa

SEQ ID NO: 6 - DNA, 21 bp, synthetic construct
  source 1..21: mol_type = other DNA
ggatggtccagtgctacacaa

SEQ ID NO: 7 - DNA, 21 bp, synthetic construct
  source 1..21: mol_type = other DNA
  modified_base 21: mod_base = OTHER; note = t-butylbenzyldA
ggatggtccagtgctacacan

SEQ ID NO: 8 - DNA, 27 bp, synthetic construct
  source 1..27: mol_type = other DNA
agaaagaggagatcttcgtacatgaga

SEQ ID NO: 9 - DNA, 27 bp, synthetic construct
  source 1..27: mol_type = other DNA
  modified_base 27: mod_base = OTHER; note = t-butylbenzyldA
agaaagaggagatcttcgtacatgagn

SEQ ID NO: 10 - DNA, 21 bp, synthetic construct
  source 1..21: mol_type = other DNA
  misc_feature 1: note = quencher attached to a nucleotide sequence
gggcataatatgtctctccca

SEQ ID NO: 11 - DNA, 21 bp, synthetic construct
  source 1..21: mol_type = other DNA
  misc_feature 1: note = quencher attached to a nucleotide sequence
  misc_feature 21: note = nucleotide attached to a C3 spacer
gggcataatatgtctctccca

SEQ ID NO: 12 - DNA, 22 bp, synthetic construct
  source 1..22: mol_type = other DNA
actgcgcttttgtcaatgtcta

SEQ ID NO: 13 - DNA, 22 bp, synthetic construct
  source 1..22: mol_type = other DNA
  modified_base 22: mod_base = OTHER; note = t-butylbenzyldA
actgcgcttttgtcaatgtctn

SEQ ID NO: 14 - DNA, 26 bp, synthetic construct
  source 1..26: mol_type = other DNA
ctgctgaaatgaaggcataattttga

SEQ ID NO: 15 - DNA, 26 bp, synthetic construct
  source 1..26: mol_type = other DNA
  modified_base 26: mod_base = OTHER; note = t-butylbenzyldA
ctgctgaaatgaaggcataattttgn

SEQ ID NO: 16 - DNA, 29 bp, synthetic construct
  source 1..29: mol_type = other DNA
  misc_feature 1: note = quencher attached to a nucleotide sequence
aatggcgacaatttaacgattaccggaca

SEQ ID NO: 17 - DNA, 29 bp, synthetic construct
  source 1..29: mol_type = other DNA
  misc_feature 1: note = quencher attached to a nucleotide sequence
  misc_feature 29: note = nucleotide attached to a C3 spacer
aatggcgacaatttaacgattaccggaca

SEQ ID NO: 18 - DNA, 3018 bp, Chlamydia trachomatis
  source 1..3018: mol_type = genomic DNA
atgcctttttctttgagatctacatcattttgttttttagcttgtttgtgttcctattcgtatggattcgcgagctctcctcaagtgttaacgcctaatgtaaccactccttttaagggagacgatgtttacttgaatggagactgcgcttttgtcaatgtctatgcaggagctgaagaaggttcgattatctcagctaatggcgacaatttaacgattaccggacaaaaccatacattatcatttacagattctcaagggccagttcttcaaaattatgccttcatttcagcaggagagacacttactctgagagatttttcgagtctgatgttctcgaaaaatgtttcttgcggagaaaagggaatgatctccgggaaaaccgtgagtatttccggagcaggcgaagtgattttctgggataactccgtggggtattctcctttatctactgtgccaacctcatcatcaactccgcctgctccaacagttagtgatgctcggaaagggtctattttttctgtagagactagtttggagatctcaggcgtcaaaaaaggggtcatgttcgataataatgccgggaatttcggaacagtttttcgaggtaagaataataataatgctggtggtggaggcagtggttccgctacaccatcaagtacgacttttacagttaaaaactgtaaagggaaagtttctttcacagataacgtagcctcttgcggaggcggagtggtttataaaggcattgtgcttttcaaagacaatgaaggaggcatattcttccgagggaacacagcatacgatgatttaaggattcttgctactaatcaggatcagaatacggagacaggaggcggtggaggagttatttgctctccagatgattctgtaaagtttgaaggcaataaaggttctattgtttttgattacaactttgcaaaaggcagaggcggaagcatcctaacgaaagaattctcgcttgtagcagatgattcggttgtctttagtaacaatacagcagaaaaaggcggtggagctatttatgctcctactatcaatatgagcacgaatgggggatcgattctgtttgaaagaaaccgagctgcggaaggaggcgccatctgcgtaagcgcagcgagctctggttcgactggcaagctcactttaagcgcttctgatggggatattgttttttctgggaatatgactagtgatcgtcctggagagcgcagcgcagtaagaattttaagcagtggaacaacagttgctttgaatgcttccggattatcgaaactgatctttcatgatcctgtagtacaaaataattcagcagcggctgcatcgacaccatcaccatcttcttcttctacgcctgctgcagtcacaattaatcagtccggtaatggatctgtgatttttaccgccgagtcattgactccttcagaaaaacttcaagttcttaactctacttcaaatttcccaggaactctgactgtgtcaggaggggagttggttgtgacggaaggagctaccttaactactgggaccattacagccacctccggacgagtgactttaggatccggagcttcgttgtctgccgttgcaggtgctgcaaataataattatacttgtacagtatctaagttggggattgatttagaatcctttttaactcctaactataagactgccatactgggtgcgtatggaacagttactgttaaaaaaggctctactttagacctagtgatggagaatgaggcggaggtctatgataatccgctttttgtgggatcgctgacgattccttttgttaccctatcttctagtagtactagtaacggggttacaatgaagcgtgttactattaatgatgcagatgctgcgcactacgggtatcaaggttcttggtctgcagattggacgaaacctcccttagctcctgatgctaagggtatggtgcctcctaatacaagtaataccttatatctgacatggagacccgcttccaactacggggagtatcgattagatcctcagagaaagggggaattggtgcccaactctctttgggtagcgggatctgcattaagaacctttactaatggtttgaaagaacactatgtttctagagatgttggatttgtagcatctctgcatgctctcggggattatattctgaattatacgcaagatgatcgggatggctttttagctagatatgggggattccaggcgactgctgcctgccattacgaaaatgggtccatattcggagtggcttttggacaactatatggtcaaacaaaaagccgaatgtattactccaaggatgcagggaacatgacgatgttgtcctgtttcggaagaagttacgtagatattaaaggaacagaaactgttgtgtactgggagacagcttatggctattctgtgcacagaatgcatacgcagtattttaatgacaaaacgcagaagttcgatcattcgaaatgtcattggcacaacaataactattatgcgtttgtaggtgctgagcataatttcttagagtactgcattcctactcgtcagttcgctagagattatgagcttgcagggtttatgcgttttgagatggctggaggatggtccagtgctacacaagaaactggttccttaactagatatttcgctcgaggagcggggcataatatgtctctcccaataggaattgtagctcatgcagtttctcatgtacgaagatctcctctttctaaactgacgttaaatatgggatatagaccagacatttggcgtgtcactccacattgcaatatggaaattattgccaatggagtgaagacgcctatacaaggatctccgctagcacggcatgccttcttcttagaggttcatgatactttgtatattcatcattttggaagagcttatatgaactattcattagatgctcgtcgtcgacaaacggcacattttgtatctatgggcttgaatagaatcttttaa

(19) United States
(12) Patent Application Publication
(10) Pub. No.: US 2026/0098309 A1
(43) Pub. Date: Apr. 9, 2026

(54) COMPOSITIONS AND METHODS FOR DETECTING LYMPHOGRANULOMA VENEREUM (LGV) SEROVARS OF CHLAMYDIA TRACHOMATIS

(71) Applicant: Roche Molecular Systems, Inc., Pleasanton, CA (US)

(72) Inventors: Kyle C. Cady, Redwood City, CA (US); Ryan Chan, San Jose, CA (US); Ke Chen, Palo Alto, CA (US); Patrick Lin, San Jose, CA (US); Arrash Moghaddasi, Portola Valley, CA (US); Rayyan Sheikh, Santa Clara, CA (US); Eugene Spier, Los Altos, CA (US); Jingtao Sun, San Ramon, CA (US)

(21) Appl. No.: 18/876,087

(22) PCT Filed: Jun. 29, 2023
(86) PCT No.: PCT/EP2023/067847

§ 371 Date: Dec. 17, 2024

Related U.S. Application Data
(60) Provisional application No. 63/357,179, filed on Jun. 30, 2022.

(51) Int. Cl. C12Q 1/689

(52) U.S. Cl. CPC C12Q 1/689

(57) ABSTRACT

The present disclosure relates, in general, to the methods for the rapid detection of the presence or absence of Lymphogranuloma Venereum (LGV)-causing serovars of Chlamydia trachomatis in a biological or non-biological sample. The methods can include performing an amplification step, a hybridization step, and a detection step. Furthermore, oligonucleotide primers and probes targeting the pmpH gene for the L serovars of Chlamydia trachomatis, along with kits are provided that are designed for the detection of L serovars.

### CROSS-REFERENCE TO RELATED APPLICATIONS

This application claims priority to and the benefit of U.S. Provisional Application No. 63/357,179, filed Jun. 30, 2022, the contents of which are incorporated herein by reference in their entirety.

### SEQUENCE LISTING

The instant application contains a sequencing listing, which has been submitted electronically in ASCII format and is hereby incorporated by reference in its entirety. Said ASCII copy, created on Jun. 29, 2023 is named “P37525-WO_Sequence_Listing” and is 26.813 bytes in size.

### BACKGROUND OF THE INVENTION

The present disclosure relates to the field of molecular diagnostics, and more particularly to the detection of lymphogranuloma venereum (LGV)-causing serovars (L serovars) of Chlamydia trachomatis (C. trachomatis) by a polymerase chain reaction (PCR) assay.

Infection from C. trachomatis is the leading bacterial cause of sexually transmitted diseases worldwide, with approximately 89.1 million cases occurring annually (Bebear C., de Barbeyrac B., Clinical Microbial Infect. 2009; 15:4-10). In the United States, C. trachomatis is the most frequently reported bacterial sexually transmitted disease (STD), and prevalence is highest in persons aged≤24 years. In 2013, a total of 1,401,906 cases of C. trachomatis infection were reported to the United States Centers for Disease Control and Prevention (CDC) corresponding to a rate of 446.6 cases per 100,000 population (CDC, Sexually Transmitted Disease Surveillance 2013).

The microorganism C. trachomatis is a gram-negative, nonmotile, obligate intracellular bacterium with a unique biphasic lifecycle. A variety of infections are caused by C. trachomatis, including urethritis, cervicitis, proctitis, conjunctivitis, endometritis, and salpingitis; if left untreated, these infections may ascend to the uterus, fallopian tubes, and ovaries causing pelvic inflammatory syndrome, ectopic pregnancy, and tubal factor infertility. Reiter's syndrome (urethritis, conjunctivitis, arthritis, and mucocutaneous lesions) has also been associated with genital C. trachomatis infection. Many infections remain asymptomatic, and high numbers of infected patients may not seek care. Patients often become re-infected if their sexual partners are not treated. Infants born to infected mothers can develop conjunctivitis, pharyngitis, and pneumonia. The predominant symptoms in men and women are increased discharge and dysuria; women may also present with irregular uterine bleeding.

Serovars for C. trachomatis can be separated into 3 groups: serovars A, B and C (A-C), serovars D, E, F, G, I, J and K (D-K), and L serovars, each of which is associated with a distinct clinical manifestation of Chlamydia disease. Serovars A-C manifest as ocular infections, serovars D-K as anogenital infections, and L serovars as lymphogranuloma venereum or LGV (genital ulcers/lesions). Of note, L serovars include a plurality of distinct strains, including L1, L2, L2b and L3. It is particularly important to distinguish between the sexually transmitted serovars (D-K vs. L) because different treatment regimens are prescribed for each—typically 100 mg doxycycline/orally/twice per day/7 days for most Chlamydia infections, and 100 mg doxycycline/orally/twice per day/21 days for LGV (Sexually Transmitted Infections Treatment Guidelines, 2021. “Lymphogranuloma Venereum.” Centers for Disease Control and Prevention. https://www.cdc.gov/std/treatment-guidelines/lgv.htm, 3 Sep. 2021). The most accurate diagnosis ensures the most appropriate antibiotic therapy.

Nucleic acid sequences of polymorphic membrane protein H (pmpH) genes of 19 serovars/serovariants have been described previously, e.g., in Verweij et al., Clin Microbiol Infect (2011); 17:1717-1726—NCBI accession numbers: A (AY184155), B (AY184156), Ba (AY184157), C (AY184158), D (AY184159), Da (AY967759), E (AY184160), Swedish variant E (SW-E; FN652779), F (AY184161), G (AY184162), H (AY184163), I (AY184164), Ia (AY967760), J (AY184165), K (AY184166), L1 (AY184167), L2 (AY184168) and L3 (AY184169).

Current methods to diagnose LGV include clinical diagnosis, epidemiologic information, and LGV-specific molecular testing (i.e., Nucleic Acid Amplification Testing or NAAT); molecular testing is the only option that can definitively diagnose LGV. In current molecular diagnostics for LGV, targeting the polymorphic membrane protein H (pmpH) gene has predominated as all the LGV serovars possess a unique gap of 36 nucleotides, which is absent in the other serovars (see FIG. 1). For at least the forgoing reasons, there is a need for improved methods for specifically and selectively detecting L serovars of C. trachomatis in nucleic acid samples.

### SUMMARY OF THE INVENTION

The present invention overcomes the aforementioned drawbacks by providing methods for specifically and selectively detecting L serovars of C. trachomatis in nucleic acid samples.

In the present disclosure, PCR assays are provided to specifically detect the pmpH gene of L serovars of C. trachomatis in biological and non-biological samples. While all C. trachomatis serovars possess the pmpH gene, the pmpH gene of the L serovars includes at least two regions that enable specific and selective detection by PCR relative to the pmpH gene of non-L serovars (e.g., servars A, B, C, D, E, F, G, I, J and K). In one aspect, a first region of the pmpH gene of the L serovars has a unique 36 base pair (bp) deletion that can be utilized for molecular diagnostics. In another aspect, a second region comprising the 3′ end of the pmpH gene of the L serovars can additionally or alternatively be utilized for molecular diagnostics.

The present disclosure relates to methods for the rapid detection of the presence or absence of L serovars in a biological or non-biological sample by a real-time PCR (RT-PCR) in a single test tube. Herein, methods of detection of the L serovars comprising performing at least one cycling step, which may include an amplifying step and a hybridizing step. Furthermore, provided are primers, probes, and kits that are designed for the detection of L serovars in a single tube. The detection methods are designed to target the pmpH gene of the L serovars, which allows one to detect L serovars in a single test.

In one aspect, a method for detecting L serovar in a sample is provided, including providing a sample, performing an amplification step comprising contacting a sample with at least one set of primers designed to target a polymorphic membrane protein H (pmpH) gene of the L serovar to produce amplification products, if the L serovar is present in the sample, performing a hybridization step, comprising contacting the amplification products, if the L serovar is present in the sample, with at least one detectable probe to target the pmpH gene of the L serovar, and performing a detection step, comprising detecting the presence or absence of the amplification products, wherein the presence of the amplification products is indicative of the presence of the L serovar in the sample, and wherein the absence of the amplification products is indicative of the absence of the L serovar in the sample. Herein, the at least one set of primers and the at least one detectable probe comprise a forward primer comprising or consisting of a nucleic acid sequence selected from the group consisting of SEQ ID NOs: 1, 2, 6 and 7, or a combination thereof, a reverse primer comprising or consisting of a nucleic acid sequence selected from the group consisting of SEQ ID NOs: 3, 4, 8, and 9, or a combination thereof, and a probe comprising or consisting of a nucleic acid sequence selected from the group consisting of SEQ ID NOs: 5, 10 and 11, or a complement thereof. In some embodiments, the forward primer comprises or consists of a nucleic acid sequence selected from the group consisting of SEQ ID NOs: 1 and 2, or a combination thereof, the reverse primer comprises or consists of a nucleic acid sequence selected from the group consisting of SEQ ID NOs: 3 and 4, or a combination thereof, and the probe comprises or consists of a nucleic acid sequence of SEQ ID NO: 5, or a complement thereof. In some embodiments, the forward primer comprises or consists of a nucleic acid sequence of SEQ ID NOs: 1 and 2, or a combination thereof; the reverse primer comprises or consists of a nucleic acid sequence of SEQ ID NOs: 3; and the probe comprises or consists of a nucleic acid sequence of SEQ ID NO: 5, or a complement thereof or the forward primer comprises or consists of a nucleic acid sequence of SEQ ID NOs: 1 and 2, or a combination thereof; the reverse primer comprises or consists of a nucleic acid sequence of SEQ ID NOs: 4; and the probe comprises or consists of a nucleic acid sequence of SEQ ID NO: 5, or a complement thereof. In some embodiments, the forward primer comprises or consists of a nucleic acid sequence selected from the group consisting of SEQ ID NOs: 6 and 7, or a combination thereof; the reverse primer comprises or consists of a nucleic acid sequence selected from the group consisting of SEQ ID NOs: 8 and 9, or a combination thereof; and the probe comprises or consists of a nucleic acid sequence selected from the group consisting of SEQ ID NOs: 10 and 11, or a complement thereof. In certain embodiments, the forward primer comprises or consists of a nucleic acid sequence of SEQ ID NO: 7; the reverse primer comprises or consists of a nucleic acid sequence of SEQ ID NO: 9; and the probe comprises or consists of a nucleic acid sequence of SEQ ID NO: 11, or a complement thereof. In some embodiments, the oligonucleotide primers and/or oligonucleotide probe have 50 or fewer nucleotides. In certain embodiments, the oligonucleotide primers and/or oligonucleotide probe have 40 or fewer nucleotides (e.g., 35 or fewer nucleotides, 30 or fewer nucleotides, 25 or fewer nucleotides). In some embodiments, the hybridization step comprises contacting the amplification products with the detectable probe that is labeled with a donor fluorescent moiety and a corresponding acceptor moiety; and the detection step comprises detecting the presence or absence of fluorescence resonance energy transfer (FRET) between the donor fluorescent moiety and the acceptor moiety of the probes, wherein the presence or absence of fluorescence is indicative of the presence or absence of L serovar in the sample. In some embodiments, the amplification step employs a polymerase enzyme having 5′ to 3′ nuclease activity. In some embodiments, the sample is a biological sample. In particular embodiment, the biological sample is a vaginal swab specimen, a clinician-collected vaginal swab specimen, an endocervical swab specimen, an oropharyngeal (throat) swab specimen, or an anorectal swab specimen. In some embodiments, the amplification and the hybridization steps are repeated. Herein, the number of repetitions depends, e.g., on the nature of the sample. If the sample is a complex mixture of nucleic acids, more amplification and hybridization steps will be required to amplify the target sequence sufficient for detection. In some embodiments, the amplification and the hybridization steps are repeated at least about 20 times, but may be repeated as many as at least 25, 30, 40, 50, 60, or even 100 times. Further, detecting the presence or absence of the amplification product may be performed during or after each amplification and hybridization step, during or after every other amplification and hybridization step, during or after particular amplification and hybridization steps or during or after particular amplification and hybridization steps, in which—if present—sufficient amplification product for detection is expected. In some embodiments, the donor fluorescent moiety and the acceptor moiety, e.g., a quencher, may be within no more than 5 to 20 nucleotides (e.g., 8 or 10) of each other along the length of the probe. In some embodiments, the donor fluorescent moiety and the corresponding acceptor moiety are within no more than 8-20 nucleotides of each other on the probe. In some embodiments, the acceptor moiety is a quencher. In another embodiment, the detectable probe further includes a nucleic acid sequence that permits secondary structure formation. Such secondary structure formation generally results in spatial proximity between the first and second fluorescent moiety. According to this method, the second fluorescent moiety on the probe can be a quencher. In some embodiments, the detectable probe may be labeled with a fluorescent dye that acts as a reporter. The probe may also have a second dye that acts as a quencher. The reporter dye is measured at a defined wavelength, thus permitting detection and discrimination of the amplified pmpH gene of the L serovar. The fluorescent signal of the intact probes is suppressed by the quencher dye. During the PCR amplification step, hybridization of the probes to the specific single-stranded DNA template results in cleavage by the 5′ to 3′ nuclease activity of the DNA polymerase resulting in separation of the reporter and quencher dyes and the generation of a fluorescent signal. With each PCR cycle, increasing amounts of cleaved probes are generated and the cumulative signal of the reporter dye is concomitantly increased. Optionally, one or more additional probes (e.g., such as an internal reference control or other targeted probe (e.g., other viral nucleic acids) may also be labeled with a reporter fluorescent dye, unique and distinct from the fluorescent dye label associated with the target gene probe. In such case, because the specific reporter dyes are measured at defined wavelengths, simultaneous detection and discrimination of the amplified targets and the one or more additional probes is possible.

In another aspect, a method for detecting a lymphogranuloma venereum-causing serovar (L serovar) of Chlamydia trachomatis in a sample is provided, the method comprising: (a) performing an amplification step comprising contacting the sample with at least one set of primers designed to target a 3′ end of the polymorphic membrane protein H (pmpH) gene of the L serovar to produce amplification products, if the L serovar is present in the sample; (b) performing a hybridization step, comprising contacting the amplification products, if the L serovar is present in the sample, with at least one detectable probe to target the pmpH gene of the L serovar; and (c) performing a detection step, comprising detecting the presence or absence of the amplification products, wherein the presence of the amplification products is indicative of the presence of the L serovar in the sample, and wherein the absence of the amplification products is indicative of the absence of the L serovar in the sample, wherein the at least one set of primers is designed to target a region comprising at least a portion of the last 1000 nucleotides of the 3′ end of the pmpH gene of the L serovar. In some embodiments, the at least one set of primers is designed to target a region comprising at least a portion of the last 500 nucleotides of the 3′ end of the pmpH gene of the L serovar. In some embodiments, the at least one set of primers is designed to target a region comprising at least a portion of nucleotides 2600 to 2900 of the pmpH gene of the L1 serovar or a corresponding region of a different L serovar. In some embodiments, the at least one set of primers is designed to target a region comprising at least a portion of nucleotides 2600 to 2800 of the pmpH gene of the L1 serovar or a corresponding region of a different L serovar. In some embodiments, the at least one set of primers is designed to target a region comprising at least a portion of nucleotides 2635 to 2766 of the pmpH gene of the L1 serovar or a corresponding region of a different L serovar. Herein, the sequence of the pmpH gene of the L1 serovar may be a nucleic acid as shown in Genbank accession number HE601950 (Gene: L1440_00934; CDS: CCP62925.1; Location: 1032228 . . . 1035245, Length: 3,018 nt) and further provided as SEQ ID NO:18. Herein, the oligonucleotide primers and/or the oligonucleotide probe are selected to efficiently hybridize to the L serovars, while not hybridizing to the other serovars. In some embodiments, the oligonucleotide probe is selected to only hybridize to the L serovars, but not to all other serovars. In certain embodiments, the oligonucleotide probe is selected to hybridize to at least a portion of nucleotides 2660 to 2740 of the pmpH gene of the L1 serovar (SEQ ID NO:18) or a corresponding region of a different L serovar, more specifically to at least a portion of nucleotides 2670 to 2730 of the pmpH gene of the L1 serovar (SEQ ID NO:18) or a corresponding region of a different L serovar, or even more specifically to at least a portion of nucleotides 2680 to 2720 of the pmpH gene of the L1 serovar (SEQ ID NO:18) or a corresponding region of a different L serovar. In some embodiments, the oligonucleotide primers and/or oligonucleotide probe have 50 or fewer nucleotides. In certain embodiments, the oligonucleotide primers and/or oligonucleotide probe have 40 or fewer nucleotides (e.g., 35 or fewer nucleotides, 30 or fewer nucleotides, 25 or fewer nucleotides). In some embodiments, the hybridization step comprises contacting the amplification products with the detectable probe that is labeled with a donor fluorescent moiety and a corresponding acceptor moiety; and the detection step comprises detecting the presence or absence of fluorescence resonance energy transfer (FRET) between the donor fluorescent moiety and the acceptor moiety of the probes, wherein the presence or absence of fluorescence is indicative of the presence or absence of L serovar in the sample. In some embodiments, the amplification step employs a polymerase enzyme having 5′ to 3′ nuclease activity. In some embodiments, the sample is a biological sample. In particular embodiment, the biological sample is a vaginal swab specimen, a clinician-collected vaginal swab specimen, an endocervical swab specimen, an oropharyngeal (throat) swab specimen, or an anorectal swab specimen. In some embodiments, the amplification and the hybridization steps are repeated.

Herein, the number of repetitions depends, e.g., on the nature of the sample. If the sample is a complex mixture of nucleic acids, more amplification and hybridization steps will be required to amplify the target sequence sufficient for detection. In some embodiments, the amplification and the hybridization steps are repeated at least about 20 times, but may be repeated as many as at least 25, 30, 40, 50, 60, or even 100 times. Further, detecting the presence or absence of the amplification product may be performed during or after each amplification and hybridization step, during or after every other amplification and hybridization step, during or after particular amplification and hybridization steps or during or after particular amplification and hybridization steps, in which—if present—sufficient amplification product for detection is expected. In some embodiments, the donor fluorescent moiety and the acceptor moiety, e.g., a quencher, may be within no more than 5 to 20 nucleotides (e.g., 8 or 10) of each other along the length of the probe. In some embodiments, the donor fluorescent moiety and the corresponding acceptor moiety are within no more than 8-20 nucleotides of each other on the probe. In some embodiments, the acceptor moiety is a quencher. In another embodiment, the detectable probe further includes a nucleic acid sequence that permits secondary structure formation. Such secondary structure formation generally results in spatial proximity between the first and second fluorescent moiety. According to this method, the second fluorescent moiety on the probe can be a quencher. In some embodiments, the detectable probe may be labeled with a fluorescent dye that acts as a reporter. The probe may also have a second dye that acts as a quencher. The reporter dye is measured at a defined wavelength, thus permitting detection and discrimination of the amplified pmpH gene of the L serovar. The fluorescent signal of the intact probes is suppressed by the quencher dye. During the PCR amplification step, hybridization of the probes to the specific single-stranded DNA template results in cleavage by the 5′ to 3′ nuclease activity of the DNA polymerase resulting in separation of the reporter and quencher dyes and the generation of a fluorescent signal. With each PCR cycle, increasing amounts of cleaved probes are generated and the cumulative signal of the reporter dye is concomitantly increased. Optionally, one or more additional probes (e.g., such as an internal reference control or other targeted probe (e.g., other viral nucleic acids) may also be labeled with a reporter fluorescent dye, unique and distinct from the fluorescent dye label associated with the target gene probe. In such case, because the specific reporter dyes are measured at defined wavelengths, simultaneous detection and discrimination of the amplified targets and the one or more additional probes is possible.

In another aspect, a kit for detecting L serovar is provided. In particular provided is a kit for detecting pmpH gene of a lymphogranuloma venereum-causing serovar (L serovar) of Chlamydia trachomatis in a sample, the kit comprising amplification reagents comprising: (a) a DNA polymerase having 5′ to 3′ nuclease activity; (b) nucleotide monomers; (c) at least one pair of primers and at least one detectable probe comprising: (i) a forward primer comprising a nucleic acid sequence of SEQ ID NOs: 1, 2, 6 and 7 or any combination thereof; (ii) a reverse primer comprising a nucleic acid sequence of SEQ ID NOs: 3, 4, 8 and 9, or a combination thereof; and (iii) a detectable probe comprising a nucleic acid sequence of SEQ ID NOs: 5, 10 and 11, or a complement thereof. In some embodiments, the forward primer comprises or consists of a nucleic acid sequence selected from the group consisting of SEQ ID NOs: 1 and 2, or a combination thereof, the reverse primer comprises or consists of a nucleic acid sequence selected from the group consisting of SEQ ID NOs: 3 and 4, or a combination thereof, and the probe comprises or consists of a nucleic acid sequence of SEQ ID NO: 5, or a complement thereof. In some embodiments, the forward primer comprises or consists of a nucleic acid sequence of SEQ ID NOs: 1 and 2, or a combination thereof, the reverse primer comprises or consists of a nucleic acid sequence of SEQ ID NOs: 3; and the probe comprises or consists of a nucleic acid sequence of SEQ ID NO: 5, or a complement thereof or the forward primer comprises or consists of a nucleic acid sequence of SEQ ID NOs: 1 and 2, or a combination thereof; the reverse primer comprises or consists of a nucleic acid sequence of SEQ ID NOs: 4; and the probe comprises or consists of a nucleic acid sequence of SEQ ID NO: 5, or a complement thereof. In some embodiments, the forward primer comprises or consists of a nucleic acid sequence selected from the group consisting of SEQ ID NOs: 6 and 7, or a combination thereof; the reverse primer comprises or consists of a nucleic acid sequence selected from the group consisting of SEQ ID NOs: 8 and 9, or a combination thereof; and the probe comprises or consists of a nucleic acid sequence selected from the group consisting of SEQ ID NOs: 10 and 11, or a complement thereof. In certain embodiments, the forward primer comprises or consists of a nucleic acid sequence of SEQ ID NO: 7; the reverse primer comprises or consists of a nucleic acid sequence of SEQ ID NO: 9; and the probe comprises or consists of a nucleic acid sequence of SEQ ID NO: 11, or a complement thereof. In some embodiments, the oligonucleotide primers and/or oligonucleotide probe have 50 or fewer nucleotides. In certain embodiments, the oligonucleotide primers and/or oligonucleotide probe have 40 or fewer nucleotides (e.g., 35 or fewer nucleotides, 30 or fewer nucleotides, 25 or fewer nucleotides). In some embodiments, the kit can include probes already labeled with donor and corresponding acceptor fluorescent moieties, or can include fluorophoric moieties for labeling the probes. In some embodiments, the detectable probe is labeled with a donor fluorescent moiety and a corresponding acceptor moiety. In some embodiments, the kit may also include nucleoside triphosphates, nucleic acid polymerase, and buffers necessary for the function of the nucleic acid polymerase. In some embodiments, the kit can also include a package insert and instructions for using the primers, probes, and fluorophoric moieties to detect the presence or absence of L serovar in a sample.

In another aspect, an oligonucleotide is provided comprising or consisting of a nucleic acid sequence selected from SEQ ID NOs: 1-11, or a complement thereof. In some embodiments, the oligonucleotide may have 50 or fewer nucleotides. In certain embodiments, the oligonucleotide may have 40 or fewer nucleotides (e.g., 35 or fewer nucleotides, 30 or fewer nucleotides, 25 or fewer nucleotides). In some embodiments, the oligonucleotide may used as an oligonucleotide primer and/or as an oligonucleotide probe. In some embodiments, the oligonucleotides comprise at least one modified nucleotide, e.g., to alter nucleic acid hybridization stability relative to unmodified nucleotides. In some embodiments, the at least one modified nucleotide is selected from the group consisting of a N6-benzyl-dA, a N4-benzyl-dC, a N6-para-tert-butyl-benzyl-dA, and a N4-para-tert-butyl-benzyl-dC. Optionally, the oligonucleotides comprise at least one label and/or at least one quencher moiety. In some embodiments, the oligonucleotides include at least one conservatively modified variation. “Conservatively modified variations” or, simply, “conservative variations” of a particular nucleic acid sequence refers to those nucleic acids, which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid. In some embodiments, the oligonucleotides are provided in a set of oligonucleotides. Herein, the set of oligonucleotides may comprise: (i) a forward primer comprising or consisting of a nucleic acid sequence of SEQ ID NOs: 1, 2, 6 and 7 or any combination thereof, (ii) a reverse primer comprising or consisting of a nucleic acid sequence of SEQ ID NOs: 3, 4, 8 and 9, or a combination thereof. In certain embodiments, the set of oligonucleotides further comprises (iii) a detectable probe comprising a nucleic acid sequence of SEQ ID NOs: 5, 10 and 11, or a complement thereof. In some embodiments, the forward primer comprises or consists of a nucleic acid sequence selected from the group consisting of SEQ ID NOs: 1 and 2, or a combination thereof; and the reverse primer comprises or consists of a nucleic acid sequence selected from the group consisting of SEQ ID NOs: 3 and 4, or a combination thereof. Herein, in certain embodiments, the probe may comprise or consist of a nucleic acid sequence of SEQ ID NO: 5, or a complement thereof. In some embodiments, the forward primer comprises or consists of a nucleic acid sequence of SEQ ID NOs: 1 and 2, or a combination thereof; and the reverse primer comprises or consists of a nucleic acid sequence of SEQ ID NOs: 3. Herein, in certain embodiments, the probe may comprise or consist of a nucleic acid sequence of SEQ ID NO: 5, or a complement thereof. In some embodiments, the forward primer comprises or consists of a nucleic acid sequence of SEQ ID NOs: 1 and 2, or a combination thereof; and the reverse primer comprises or consists of a nucleic acid sequence of SEQ ID NOs: 4. Herein, in certain embodiments, the probe may comprise or consist of a nucleic acid sequence of SEQ ID NO: 5, or a complement thereof. In some embodiments, the forward primer comprises or consists of a nucleic acid sequence selected from the group consisting of SEQ ID NOs: 6 and 7, or a combination thereof; and the reverse primer comprises or consists of a nucleic acid sequence selected from the group consisting of SEQ ID NOs: 8 and 9, or a combination thereof. Herein, in certain embodiments, the probe may comprise or consist of a nucleic acid sequence selected from the group consisting of SEQ ID NOs: 10 and 11, or a complement thereof. In certain embodiments, the forward primer comprises or consists of a nucleic acid sequence of SEQ ID NO: 7; and the reverse primer comprises or consists of a nucleic acid sequence of SEQ ID NO: 9. Herein, in certain embodiments, the probe may comprise or consist of a nucleic acid sequence of SEQ ID NO: 11, or a complement thereof. In some embodiments, at least one of the primers and/or detectable probe that target the pmpH gene of the L serovar comprises at least one modified nucleotide as detailed out above.

In another aspect, the determination of the presence of an L serovar nucleic acid of Chlamydia trachomatis in the methods and kits provided above may be combined with the determination of the presence of all serovar nucleic acids of Chlamydia trachomatis. Herein, the above disclosed oligonucleotide sets used in the methods and kits above may be combined with an oligonucleotide set comprising at least a forward and a reverse oligonucleotide primer and at least one oligonucleotide probe that amplify and detect all serovars of Chlamydia trachomatis (CT). In some embodiments, the oligonucleotide set comprises at least one pan-CT forward primer comprising a nucleic acid sequence selected from the group consisting of SEQ ID NOs: 12 and 13, or a combination thereof; at least one pan-CT reverse primer comprising a nucleic acid sequence selected from the group consisting of SEQ ID NOs: 14 and 15, or a combination thereof, and at least one pan-CT probe comprising a nucleic acid sequence selected from the group consisting of SEQ ID NOs: 16 and 17, or a complement thereof. In certain embodiments, the amplification, hybridization and detection step using the set of pan-CT oligonucleotides is performed using a second portion of the sample in a parallel reaction. In alternative embodiments the amplification, hybridization and detection step using the set of pan-CT oligonucleotides is performed in a multiplex reaction in the same vial as the reaction for determining the L serovars. Herein, the fluorophore (or the donor-acceptor pair) for labeling the pan-CT oligonucleotide probe is distinct from the fluorophore (or the donor-acceptor pair) for labeling the L serovar specific oligonucleotide probe. More specifically, the hybridization step may comprise contacting the amplification products with two or more probes that are each labeled with a distinct donor fluorescent moiety (e.g., a first and a second donor fluorescent moiety) and a corresponding acceptor moiety; and the detection step comprises detecting the presence or absence of fluorescence resonance energy transfer (FRET) between the donor fluorescent moiety (e.g., the first and a second donor fluorescent moiety) and the acceptor moiety of the probes for both distinct donor fluorescent moieties and the corresponding acceptor moieties, wherein the presence or absence of fluorescence is indicative of the presence or absence of the Chlamydia trachomatis (CT) L serovars and/or the presence or absence of any of the Chlamydia trachomatis (CT) serovars in the sample.

The present disclosure also provides methods of detecting the presence or absence of the L serovar (i.e., pmpH gene having the 36 base deletion) nucleic acid, in a biological sample from an individual. These methods can be employed to detect the presence or absence of L serovar or L serovar nucleic acid in biological samples such as vaginal swab specimens, clinician-collected vaginal swab specimens, endocervical swab specimens, oropharyngeal (throat) swab specimens, and anorectal swab specimens, or other biological materials believed to have L serovar present, for use in diagnostic testing. Additionally, the same test may be used by someone experienced in the art to assess other sample types to detect L serovar nucleic acid. Such methods generally include performing at least one cycling step, which includes an amplification step and either a detectable probe hybridization step or a dye-binding step. Typically, the amplification step includes contacting the sample with at least one pair of oligonucleotide primers to produce one or more amplification products if a nucleic acid molecule is present in the sample, the probe hydridization step includes contacting the amplification product with one or more detectable probes specific for the amplification product, and the dye-binding step includes contacting the amplification product with a double-stranded DNA binding dye. Such methods also include detecting the presence or absence of binding of the double-stranded DNA binding dye into the amplification product, wherein the presence of binding is indicative of the presence of L serovar nucleic acid in the sample, and wherein the absence of hybridization is indicative of the absence of L serovar or L serovar nucleic acid in the sample. A representative double-stranded DNA binding dye is ethidium bromide. Other nucleic acid-binding dyes include DAPI, Hoechst dyes, PicoGreen®, RiboGreen®, OliGreen®, and cyanine dyes such as YO-YO® and SYBR® Green. In addition, such methods also can include determining the melting temperature between the amplification product and the double-stranded DNA binding dye, wherein the melting temperature confirms the presence or absence of L serovar or L serovar nucleic acid.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 is a sequence alignment of C. trachomatis serovars A-C/K/J compared to serovars L1/L2/L3 showing the unique 36-bp deletion present only in serovars L1/L2/L3. An example of how a primer/probe detection set can be designed to specifically detect the L serovars.

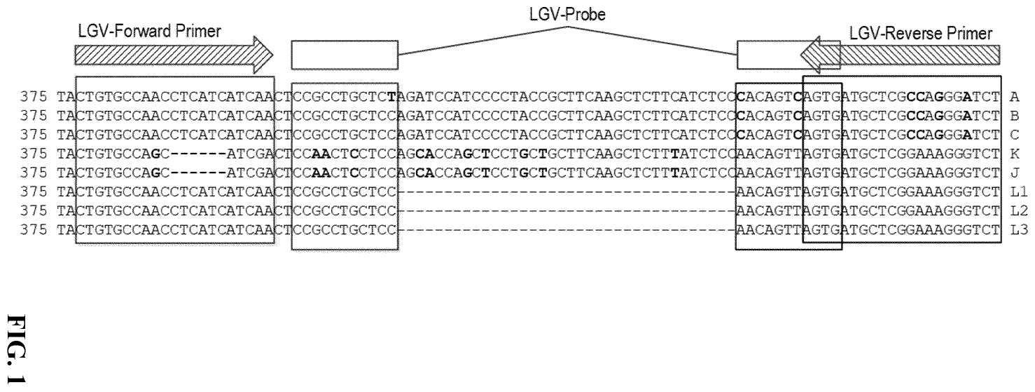

FIG. 2 is a sequence alignment of the pmpH gene of C. trachomatis serovars A, B, C, D, E, F, G, Ia, J and K as compared to serovars L1, L2, L2b and L3 showing a first region comprising the unique 36-bp deletion present only in the L serovars (indicated as R1) and a second region located at the 3′ end of the pmpH gene (indicated as R2). The x-axis indicates the distance along the pmpH gene in nucleotides in the 5′ to 3′ direction. The y-axis shows, from top to bottom, a chart illustrating the consensus identity (CID) of the listed serovars, followed by serovars L2, L2b, L3, L1, A, B, C, F, G, E, D, Ia, K and J. Triangles indicate the binding locations of the primers and probes of the present disclosure (see Table 1).

### DETAILED DESCRIPTION

### I. Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art; and (v) where ranges are provided, endpoints are included.

As used herein, the term “amplifying” or “amplification” refers to the process of synthesizing nucleic acid molecules that are complementary to one or both strands of a template nucleic acid molecule (e.g. pmpH gene from L serovars of C. trachomatis). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level, and/or form of one is correlated with that of the other. For example, a particular entity (e.g., polypeptide, genetic signature, metabolite, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises or consists of an organism, such as an animal or human. In some embodiments, a biological sample is comprises or consists of biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; other body fluids, secretions, and/or excretions; and/or cells therefrom, etc. In some embodiments, a biological sample is comprises or consists of cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

The term “complement thereof” refers to nucleic acid that is both the same length as, and exactly complementary to, a given nucleic acid.

Comprising: A composition or method described herein as “comprising” one or more named elements or steps is open-ended, meaning that the named elements or steps are essential, but other elements or steps may be added within the scope of the composition or method. It is to be understood that composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Designed: As used herein, the term “designed” refers to an agent (i) whose structure is or was selected by the hand of man; (ii) that is produced by a process requiring the hand of man; and/or (iii) that is distinct from natural substances and other known agents.

Determine: Those of ordinary skill in the art, reading the present specification, will appreciate that “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The term “hybridizing” or “hybridization” refers to the annealing of one or more probes to an amplification product. Hybridization conditions typically include a temperature that is below the melting temperature of the probes but that avoids non-specific hybridization of the probes.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolo-pyrimidine analog, a pseudo-dU, a nitro pyrrole, a nitro indole, 2′-O-methyl Ribo-U, 2′-O-methyl Ribo-C, an N4-ethyl-dC, an N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference. Examples of these types of nucleic acid modifications are described in, e.g., U.S. Pat. No. 6,001,611, which is incorporated herein by reference.

The term “primer” is used herein as known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′-OH group whereto further “nucleotides” may be attached by a template-dependent DNA polymerase establishing 3′ to 5′ phosphodiester linkage whereby deoxynucleoside triphosphates are used and whereby pyrophosphate is released. Therefore, there is—except possibly for the intended function—no fundamental difference between a “primer”, an “oligonucleotide”, or a “probe”.

Sample: As used herein, the term “sample” refers to a substance that is or contains a composition of interest for qualitative and or quantitative assessment. In some embodiments, a sample is a biological sample (i.e., comes from a living thing (e.g., cell or organism). In some embodiments, a sample is from a geological, aquatic, astronomical, or agricultural source. In some embodiments, a source of interest comprises or consists of an organism, such as an animal or human. In some embodiments, a sample for forensic analysis is or comprises biological tissue, biological fluid, organic or non-organic matter such as, e.g., clothing, dirt, plastic, water. In some embodiments, an agricultural sample, comprises or consists of organic matter such as leaves, petals, bark, wood, seeds, plants, fruit, etc.

Specificity: As used herein, the term “specificity” means the preference of an enzyme for a specific substrate.

Selectivity: As used herein, the term “selectivity” means the preference of an enzyme for one specific substrate over another.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result.

The term “substantially” is therefore used herein to capture the potential lack of completeness inherent in many biological and chemical phenomena.

Synthetic: As used herein, the word “synthetic” means produced by the hand of man, and therefore in a form that does not exist in nature, either because it has a structure that does not exist in nature, or because it is either associated with one or more other components, with which it is not associated in nature, or not associated with one or more other components with which it is associated in nature.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T rubens, Bacillus stearothermophilus, and Methanothermus fervidus. Nonetheless, polymerases that are not thermostable also can be employed in PCR assays provided the enzyme is replenished.

The term “5′ to 3′ nuclease activity” refers to an activity of a nucleic acid polymerase, typically associated with the nucleic acid strand synthesis, whereby nucleotides are removed from the 5′ end of nucleic acid strand.

### II. Detailed Description of Certain Embodiments

Diagnosis of a lymphogranuloma venereum (LGV)-causing serovar (L serovar) of C. trachomatis infection by nucleic acid amplification provides a method for rapidly and accurately detecting the bacterial infection. An RT-PCR assay for detecting L serovars in a sample is described herein. Primers and probes for detecting the pmpH gene of the L serovars of C. trachomatis are provided, as are articles of manufacture or kits containing such primers and probes. The increased sensitivity of RT-PCR for detection of L serovars compared to other methods, as well as the improved features of RT-PCR including sample containment and real-time detection of the amplified product, make feasible the implementation of this technology for routine diagnosis of infections by an LGV-causing serovar of C. trachomatis in the clinical laboratory.

The L serovars of C. trachomatis are emerging pathogens in the industrial world for men who have sex with men, often in association with HIV co-infection. The L serovars cause acute illness, may persist for extended periods and, if untreated, may facilitate the spread of HIV. Clinical care, surveillance and research are severely hindered by the lack of widely available, rapid, standardized tests for the diagnosis of LGV.

Recently, a number of PCR-based test systems were published facilitating the identification of the LGV-associated L serovars. Some of these dealt with the detection and typing of nearly all serovars of C. trachomatis in a given sample. The discrimination of L serovars from the other serovars of C. trachomatis is of major clinical importance as different antibiotic treatment regimens are required. Detection is often based on amplification of the pmpH gene, in which the unique deletion in the L serovars facilitates the design of an L serovar-specific PCR.

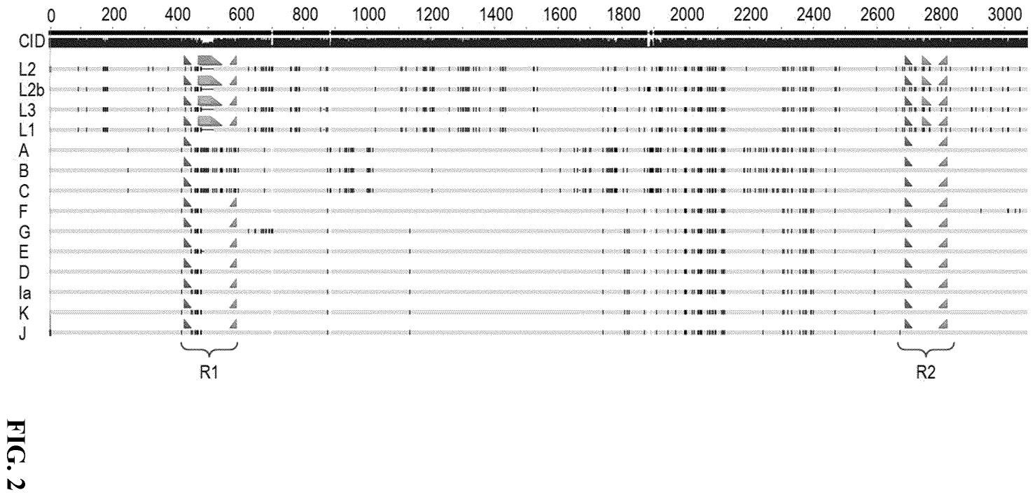

The present disclosure provides methods to detect L serovars of C. trachomatis by amplifying, for example, a first region or a second region of the pmpH nucleic acid sequence from L serovars (see FIG. 2). Nucleic acid sequences of the pmpH gene from various serovars of C. trachomatis are available (e.g., GenBank Accession No. AY184167 from serovar L1, AY184168 from serovar L2, AY184169 for serovar L3). Moreover, as indicated above, the sequence of the pmpH gene of the L1 serovar may be a nucleic acid as shown in Genbank accession number HE601950 (Gene: L1440_00934; CDS: CCP62925.1; Location: 1032228 . . . 1035245, Length: 3,018 nt). Specifically, primers and probes to amplify and detect the pmpH gene from L serovar nucleic acid molecule targets are provided by the embodiments in the present disclosure.

The present disclosure is, at least in part, based on the surprising discovery that the pmpH gene offers at least two regions useful for selectively and specifically detecting L serovars of C. trachomatis relative to other non-LGV causing serovars. Herein as shown in FIG. 2, region R1, which is the region 400-600 nucleotides from the 5′-end of pmpH gene, comprises a 36 bp deletion in L serovars (marked as grey box in the L serovars), while all other serovars lack this deletion. Hence, in a first aspect a set of oligonucleotide primers is positioned to hybridize to the R1 region in order to amplify this target region (grey triangles in FIG. 2), while an oligonucleotide probe is selected that hybridizes across the deletion site. Using this setup, the oligonucleotide probe spanning the deletion site exclusively hybridizes to the deletion site present only in the in L serovars and therefore, signal is only detected for L serovars (L1, L2, L2b, L3).

In a second aspect, surprisingly a second region, region R2 as shown FIG. 2, was identified, which is a region close to the 3′-end of the pmpH gene and 2600 to 2900 nucleotides from the 5′-end of pmpH gene (in some embodiments, 2600 to 2800 nucleotides from the 5′-end of pmpH gene). Herein, identification of the L serovars does not rely on the specific determination of a deletion only present in the L serovars. Rather, region R2 provides a variety of nucleotides that are differing between the sequences of L serovars and all other serovars. Herein, the oligonucleotide primers and/or the oligonucleotide probe are selected to efficiently hybridize to the L serovars, while not hybridizing to the other serovars. In particular, the oligonucleotide probe may be selected to only hybridize to the L serovars, but not to all other serovars (see FIG. 2 triangles indicating beneficial hybridization sites for the oligonucleotide primer set and/or the oligonucleotide probe). Herein, the oligonucleotide probe may be selected to hybridize to at least a portion of nucleotides 2660 to 2740 of the pmpH gene of the L1 serovar or a corresponding region of a different L serovar, more specifically to at least a portion of nucleotides 2670 to 2730 of the pmpH gene of the L1 serovar or a corresponding region of a different L serovar, or even more specifically to at least a portion of nucleotides 2680 to 2720 of the pmpH gene of the L1 serovar or a corresponding region of a different L serovar.

TABLE 1
Primers and Probes directed to the pmpH gene of L serovars
of C. trachomatis

| SEQ ID | | | |
|---|---|---|---|
| NO. | Name | Description | Sequence |
| 1 | SEGP3457 | F primer | CCGTGGGGTATTCTCCTTTATCTA |
| 2 | SEGP3456 | F primer | CTCCGTGGGGTATTCTCCTTTA |
| 3 | SEGP3460 | R primer | GAGATCTCCAAACTAGTCTCTACAGAA |
| 4 | SEGP3462 | R primer | TGACGCCTGAGATCTCCAAA |
| 5 | SEGP3434 | probe | /FAM/TCAACTCCG/ZEN/CCTGCTCCAACA |
| | | | GTTAGTGATGCTCGGAA/3IABKFQ/ |
| 6 | LGVpmpHF1 | F primer | GGATGGTCCAGTGCTACACAA |
| 7 | LGVpmpHF2 | F primer | GGATGGTCCAGTGCTACACA<t_BB_dA> |
| 8 | LGVpmpHR1 | R primer | AGAAAGAGGAGATCTTCGTACATGAGA |
| 9 | LGVpmpHR2 | R primer | AGAAAGAGGAGATCTTCGTACATGAG |
| | | | <t_BB_dA> |
| 10 | LGVpmpHP1 | probe | /F/AGGAGCG/Q/GGGCATAATATGTCTCTCCCA |
| 11 | LGVpmpHP2 | probe | /FAM Thr/AGGAGCG/BHQ2/GGGCATAATATG |
| | | | TCTCTCCCA/Spc_C3/ |
| 12 | panCTpmpHF1 | F primer | ACTGCGCTTTTGTCAATGTCTA |
| 13 | panCTpmpHF2 | F primer | ACTGCGCTTTTGTCAATGTCT/t_BB_dA/ |
| 14 | panCTpmpHR1 | R primer | CTGCTGAAATGAAGGCATAATTTTGA |
| 15 | panCTpmpHR2 | R primer | CTGCTGAAATGAAGGCATAATTTTG/t_BB_dA/ |
| 16 | panCTpmpHP1 | probe | /F/TCAGCT/Q/AATGGCGACAATTTAACGATTA |
| | | | CCGGACA |
| 17 | panCTpmpHP2 | probe | /HEX Thr/TCAGCT/BHQ2/AATGGCGACAATTT |
| | | | AACGATTACCGGACA/Spc_C3/ |

As shown in Table 1, F primer=forward primer; R primer=reverse primer; F/FAM/HEX Po-1=dye; Q/ZEN/BHQ2=quencher; 3IABkFQ=blocker; t_BB_dA=t-butylbenzyldA; Spc_C3=3′ C3 spacer.

More specifically as shown in Table 1, suitable primer and probe oligonucleotides are provided each include a nucleic acid with a sequence selected from SEQ ID NOs: 1-11, a substantially identical variant thereof in which the variant has at least, e.g., 80%, 90%, or 95% sequence identity to one of SEQ ID NOs: 1-11, or a complement of SEQ ID NOs: 1-11, and the variant. Herein, primer and probe oligonucleotides hybridizing to region R1 are provided each including a nucleic acid with a sequence selected from SEQ ID NOs: 1-5, a substantially identical variant thereof in which the variant has at least, e.g., 80%, 90%, or 95% sequence identity to one of SEQ ID NOs: 1-5, or a complement of SEQ ID NOs: 1-5, and the variant. Further, primer and probe oligonucleotides hybridizing to region R2 are also provided each including a nucleic acid with a sequence selected from SEQ ID NOs: 6-11, a substantially identical variant thereof in which the variant has at least, e.g., 80%, 90%, or 95% sequence identity to one of SEQ ID NOs: 6-11, or a complement of SEQ ID NOs: 6-11, and the variant.

In one embodiment, the sets of primers and probes described in Table 1 are used in order to provide for detection of one or more L serovars of C. trachomatis in a biological sample suspected of containing said one or more L serovars. The sets of primers and probes may comprise or consist the primers and probes specific for the pmpH gene from L serovar nucleic acid sequences, comprising or consisting of the nucleic acid sequences of SEQ ID NOs: 1-11.

A functionally active variant of any of the primers and/or probes of SEQ ID NOs: 1-11 may be identified by using the primers and/or probes in the disclosed methods. A functionally active variant of a primer and/or probe of any of the SEQ ID NOs: 1-11 pertains to a primer and/or probe that provides a similar or higher specificity and sensitivity in the described method or kit as compared to the respective sequence of SEQ ID NOs: 1-11.

The variant may, for example, vary from the sequence of SEQ ID NOs: 1-11 by one or more nucleotide additions, deletions or substitutions such as one or more nucleotide additions, deletions or substitutions at the 5′ end and/or the 3′ end of the respective sequence of SEQ ID NOs: 1-11. As detailed above, a primer (and/or probe) may be chemically modified; That is, a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, for example, a 7-desazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

Moreover, in some applications it may be beneficial to combine the determination of the presence of an L serovar nucleic acid of Chlamydia trachomatis with the determination of the presence of all serovar nucleic acids of Chlamydia trachomatis. This approach may be specifically beneficial when using the setup of FIG. 2 discussed above by either detecting the deletion in L serovars in region R1 using a deletion-spanning probe or detecting the L serovars using oligonucleotide primers and/or an oligonucleotide probe hybridizing within region R2 of the L serovars, but not hybridizing to the other serovars. Herein, signal is only detected for the presence of L serovars, while no signal is detected for the presence of all other serovars. However, the lack of signal could also be caused by a failure of the amplification/detection run and could thus lead to false negative results. For this, oligonucleotide primers and probes are also provided herein, that allow for the determination of all serovars of Chlamydia trachomatis (CT). As shown in Table 1, an exemplary oligonucleotide set is provided comprising at least one pan-CT forward primer comprising a nucleic acid sequence selected from the group consisting of SEQ ID NOs: 12 and 13, or a combination thereof; at least one pan-CT reverse primer comprising a nucleic acid sequence selected from the group consisting of SEQ ID NOs: 14 and 15, or a combination thereof, and at least one pan-CT probe comprising a nucleic acid sequence selected from the group consisting of SEQ ID NOs: 16 and 17, or a complement thereof. Disclosed herein is that the amplification and detection reaction using pan-CT oligonucleotides may be performed using a second portion of the sample in a parallel reaction. However, it is also possible to perform the amplification and detection reaction using pan-CT oligonucleotides in a multiplex reaction in the same vial as the reaction for determining the L serovars. Herein, the fluorophore (or the donor-acceptor pair) for labeling the pan-CT oligonucleotide probe is distinct from the fluorophore (or the donor-acceptor pair) for labeling the L serovar specific oligonucleotide probe. More specifically, the hybridizing step may comprise contacting the amplification products with the two or more probes that are each labeled with a distinct donor fluorescent moiety (e.g., a first and a second donor fluorescent moiety) and a corresponding acceptor moiety; and the detecting step comprises detecting the presence or absence of fluorescence resonance energy transfer (FRET) between the donor fluorescent moiety (e.g., the first and a second donor fluorescent moiety) and the acceptor moiety of the probes for both distinct donor fluorescent moieties and the corresponding acceptor moieties, wherein the presence or absence of fluorescence is indicative of the presence or absence of the Chlamydia trachomatis (CT) L serovars and/or the presence or absence of any of the Chlamydia trachomatis (CT) serovars in the sample.

Oligonucleotides including modified oligonucleotides and oligonucleotide analogs that amplify a nucleic acid molecule from the pmpH gene from L serovar nucleic acid sequences can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length).

In addition to a set of primers, the methods may use one or more probes in order to detect the presence or absence of L serovars. The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”, in the present case to a pmpH gene from L serovar nucleic acids. A “probe” can be referred to as a “detection probe” meaning that it detects the target nucleic acid.

In some embodiments, the probe can be labeled with at least one fluorescent label. In one embodiment, the probe directed to the pmpH gene from L serovars can be labeled with a donor fluorescent moiety, for example, a fluorescent dye, and a corresponding acceptor fluorescent moiety, for example, a quencher. In one embodiment, the probe comprises or consists of a fluorescent moiety and the nucleic acid sequences comprise or consist of SEQ ID NO: 5. In another embodiment, the probe comprises or consists of a fluorescent moiety and the nucleic acid sequences comprise or consist of SEQ ID NO: 11.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

Constructs containing target gene nucleic acid molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described target nucleic acid sequences (e.g., SEQ ID NOs: 1-4). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min).

If the double-stranded template nucleic acid is denatured by heat, the reaction mixture is allowed to cool to a temperature that promotes annealing of each primer to its target sequence on the described target gene nucleic acid molecules. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized; That is, a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min).

PCR assays can employ template nucleic acid such as RNA or DNA (cDNA). The template nucleic acid need not be purified; it may be a minor fraction of a complex mixture, such as C. trachomatis nucleic acid contained in human cells. C. trachomatis nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers (e.g., SEQ ID NOs: 1-4 and 6-9) are combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target gene nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, for example, on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, an oligonucleotide probe can contain a donor fluorescent moiety and a corresponding quencher, which may or not be fluorescent, and which dissipates the transferred energy in a form other than light. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ nuclease activity of, for example, a Taq Polymerase such that the fluorescent emission of the donor fluorescent moiety is no longer quenched. Exemplary probes for this purpose are described in, for example, U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Cal.), Iowa Black™ (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the C. trachomatis target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range. As used herein with respect to donor and corresponding acceptor fluorescent moieties “corresponding” refers to an acceptor fluorescent moiety having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced there between.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, Oreg.) or Sigma Chemical Co. (St. Louis, Mo.).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide which contains an amino linker (e.g., C6-amino phosphoramidites available from ABI (Foster City, Calif) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, Mass.)), amide-linkers (fluorescein-NHS-ester-derived, such as CX-fluorescein-CPG from BioGenex (San Ramon, Calif.)), or 3′-amino-CPGs that require coupling of a fluorescein-NHS-ester after oligonucleotide synthesis.

Detection of L Serovars of C. trachomatis

The present disclosure provides methods for detecting the presence or absence of L serovars of C. trachomatis in a biological or non-biological sample. Methods provided avoid problems of sample contamination, false negatives, and false positives. The methods include performing at least one cycling step that includes amplifying a portion of the pmpH gene from L serovar target nucleic acid molecules from a sample using at least one pair of primers, and a FRET detecting step. Multiple cycling steps are performed, preferably in a thermocycler. Methods can be performed using gene primers and probes to detect the presence of pmpH gene from L serovars, and that indicates the presence of one or more L serovars in the sample.

As described herein, amplification products can be detected using labeled hybridization probes that take advantage of FRET technology. One FRET format utilizes TaqMan® technology to detect the presence or absence of an amplification product, and hence, the presence or absence of one or more L serovars. TaqMan® technology utilizes one single-stranded hybridization probe labeled with, e.g., one fluorescent dye and one quencher, which may or may not be fluorescent. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for detecting the presence or absence of L serovars in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules. If amplification of target nucleic acid occurs and an amplification product is produced, the step of hybridizing results in a detectable signal based upon FRET between the members of the pair of probes.

Generally, the presence of FRET indicates the presence of one or more L serovars in the sample, and the absence of FRET indicates the absence of L serovars in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of an infection of an L serovar of C. trachomatis.

Representative biological samples that can be used in practicing the methods include, but are not limited to blood, plasma, serum, liver samples, dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release nucleic acid or in some cases, the biological sample can be contacted directly with the PCR reaction components and the appropriate oligonucleotides.

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the probes from the respective amplification products can confirm the presence or absence of target nucleic acid in the sample.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next.

Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used. When double-stranded DNA binding dyes are used, a melting curve analysis is usually performed for confirmation of the presence of the amplification product.

It is understood that the embodiments of the present disclosure are not limited by the configuration of one or more commercially available instruments.

### Articles of Manufacture/Kits

Embodiments of the present disclosure further provide for articles of manufacture or kits to detect L serovars. An article of manufacture can include primers and probes used to detect L serovars, together with suitable packaging materials. Representative primers and probes for detection of the pmpH gene of L serovars are capable of hybridizing to the target nucleic acid molecule. In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to the target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the target gene probes. Examples of suitable FRET donor fluorescent moieties and corresponding acceptor fluorescent moieties are provided above.

Articles of manufacture can also contain a package insert or package label having instructions thereon for using the primers and probes directed to the pmpH gene from L serovars to detect L serovars in a sample. Articles of manufacture may additionally include reagents for carrying out the methods disclosed herein (e.g., buffers, polymerase enzymes, co-factors, or agents to prevent contamination). Such reagents may be specific for one of the commercially available instruments described herein.

Embodiments of the present disclosure will be further described in the following examples, which do not limit the scope of the invention described in the claims.

Notably, the embodiments of the methods according to the present disclosure can include one or more additional steps or omit one or more of the described steps of the methods. In general, the methods can be modified in any suitable way that still enables the outcome of selectively and specifically detecting one or more L serovars of C. trachomatis in a sample. Yet other variations of the methods that fall within the scope of the present disclosure will be apparent from the additional examples and description included herein.

### EXAMPLES

The following Examples are meant to be illustrative and are not intended to be limiting in any way.

### Example 1

The present example describes PCR Experimental Conditions for RT-PCR detection of the pmpH gene target. Detection by RT-PCR of the pmpH gene target was performed using the Cobas® 6800/8800 systems (Roche Molecular Systems, Inc., Pleasanton, CA). The final concentrations of the amplification reagents and the thermoprofile used for PCR amplification reaction are show in

TABLE 2
PCR Reagents and Thermoprofile
Mg Reagent (R1)

| Reagent | Test Conc | 1 rxn (uL) |
|---|---|---|
| Mn Reagent [R1] | 3.3 mM | 10.0 |
| Elution Buffer | — | 14.1 |
| Enzyme (Z05D) | — | 0.225 |
| IC | — | 5.0 |
| | Total | 29.4 |

Master Mix (R2) - Universal

| Reagent | Test Conc | 1 rxn (uL) |
|---|---|---|
| Bulk Cobas 6800/8800 MMx-R2 Buffer | — | 12 |
| Nuclease Free Water | — | 1.94 |
| UNG | — | 0.2 |
| NTQ21-46A - Aptamer | — | 0.056 |
| | Total | 14.2 |

PCR Thermal Cycling Parameters

| | | Temp | Hold Time | Ramp |
|---|---|---|---|---|
| Step | Cycles | (° C.) | (mm:ss) | (° C./sec) |
| Pre-PCR | 1 | 50 | 02:00 | 4.4 |
| | | 94 | 00:05 | 4.4 |
| | | 55 | 02:00 | 2.2 |
| | | 60 | 06:00 | 4.4 |
| | | 65 | 04:00 | 4.4 |
| First | 5 | 95 | 00:05 | 4.4 |
| Measurement | | 55.0 | 00:30 | 2.2 |
| Second | 45 | 91.0 | 00:05 | 4.4 |
| Measurement | | 58.0 | 00:25 | 2.2 |
| Post | 1 | 40 | 02:00 | 2.2 |

As shown in Table 2, Test Conc=test concentration; rxn=reaction; Temp=temperature; mm=minutes; ss=seconds; sec=second.

The pre-PCR program comprised initial denaturing and incubation at 55° C., 60° C. and 65° C. for reverse transcription of RNA templates. Incubating at three temperatures combines the advantageous effects that at lower temperatures slightly mismatched target sequences (such as genetic variants of an organism) are also transcribed, while at higher temperatures the formation of RNA secondary structures is suppressed, thus leading to a more efficient transcription. PCR cycling was divided into two measurements, wherein both measurements apply a one-step setup (combining annealing and extension). The first 5 cycles at 55° C. allow for an increased inclusivity by pre-amplifying slightly mismatched target sequences, whereas the 45 cycles of the second measurement provide for an increased specificity by using an annealing/extension temperature of 58° C.

### Example 2

Results of PCR assays using a set of primers and probe for detecting L serovars of C. trachomatis are shown in Table 3. Master mix (MMx) 1 used the forward primer of SEQ ID NO:1, the reverse primer of SEQ ID NO: 3 and the probe of SEQ ID NO: 5. MMx 2 used the forward primer of SEQ ID NO: 2, the reverse primer of SEQ ID NO: 4 and the probe of SEQ ID NO: 5. MMx 3 used the forward primer of SEQ ID NO:7, the reverse primer of SEQ ID NO:9, and the probe of SEQ ID NO:11. Template plasmids carrying consensus nucleic acid sequences for either serovars L1, L2, and L3 or serovars A, B, and C, serovars D, E, F, G, H, and I and serovars J, and K were tested at concentrations from 1.00E+01 copies per reaction to 1.00E+07 copies per reaction. Initial testing demonstrated that these primer/probe pairings produced good results in terms of sensitivity and specificity for the L serovars (i.e., serovars L1, L2 and L3).

TABLE 3
PCR Assay Results

| | | Inclusivity | | | | | | | |
|---|---|---|---|---|---|---|---|---|---|
| | | Plasmid | | Exclusivity Plasmids | | | | | |
| | | Serovars | | Serovars | | Serovars | | Serovars | |
| | | L1, L2, L3 | | A, B, C | | D, E, F, G, H, I | | J, K | |
| | Test Conc | Avg | Avg | Avg | Avg | Avg | Avg | Avg | Avg |
| MMx | (cp/rxn) | Ct | RFI | Ct | RFI | Ct | RFI | Ct | RFI |
| 1 | 1.00E+07 | 13.7 | 7.4 | — | — | — | — | — | — |
| | 1.00E+06 | 17.1 | 7.3 | 27.6 | 3.6 | NaN | 1.2 | NaN | 1.2 |
| | 1.00E+05 | 20.6 | 7.2 | — | — | — | — | — | — |
| | 1.00E+04 | 23.9 | 7.1 | — | — | — | — | — | — |
| | 1.00E+03 | 27.4 | 6.9 | — | — | — | — | — | — |
| | 1.00E+02 | 30.7 | 6.5 | — | — | — | — | — | — |
| | 1.00E+01 | 34.3 | 4.9 | — | — | — | — | — | — |
| 2 | 1.00E+07 | 13.0 | 7.3 | — | — | — | — | — | — |
| | 1.00E+06 | 16.4 | 7.2 | 29.5 | 2.8 | 26.6 | 1.1 | 26.6 | 1.2 |
| | 1.00E+05 | 19.8 | 7.2 | — | — | — | — | — | — |
| | 1.00E+04 | 23.2 | 7.1 | — | — | — | — | — | — |
| | 1.00E+03 | 27.0 | 6.8 | — | — | — | — | — | — |
| | 1.00E+02 | 30.1 | 6.4 | — | — | — | — | — | — |
| | 1.00E+01 | 33.3 | 5.4 | — | — | — | — | — | — |
| 3 | 1.00E+07 | 14.2 | 9.7 | — | — | — | — | — | — |
| | 1.00E+06 | 17.7 | 9.6 | NaN | 1.3 | NaN | 1.3 | NaN | 1.3 |
| | 1.00E+05 | 21.0 | 9.6 | — | — | — | — | — | — |
| | 1.00E+04 | 24.4 | 9.8 | — | — | — | — | — | — |
| | 1.00E+03 | 27.8 | 9.9 | — | — | — | — | — | — |
| | 1.00E+02 | 31.1 | 9.9 | — | — | — | — | — | — |
| | 1.00E+01 | 33.5 | 9.8 | — | — | — | — | — | — |

With reference to Table 3, NaN=Not a Number; Conc=concentration; Avg=average; cp/rxn=copies per reaction; Ct=cycle threshold; RFI=relative fluorescence intensity.

The present invention is presented in several varying embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the following description, numerous specific details are recited to provide a thorough understanding of embodiments of the system. One skilled in the relevant art will recognize, however, that the system and method may both be practiced without one or more of the specific details, or with other methods, components, materials, and so forth. In other instances, well-known structures, materials, or operations are not shown or described in detail to avoid obscuring aspects of the invention. Accordingly, the foregoing description is meant to be exemplary, and does not limit the scope of present inventive concepts.

Each reference identified in the present application is herein incorporated by reference in its entirety.

1. A method for detecting a lymphogranuloma venereum-causing serovar (L serovar) of Chlamydia trachomatis in a sample, the method comprising:
(a) performing an amplification step comprising contacting the sample with at least one set of primers designed to target a polymorphic membrane protein H (pmpH) gene of the L serovar to produce amplification products, if the L serovar is present in the sample;
(b) performing a hybridization step, comprising contacting the amplification products, if the L serovar is present in the sample, with at least one detectable probe to target the pmpH gene of the L serovar; and
(c) performing a detection step, comprising detecting the presence or absence of the amplification products, wherein the presence of the amplification products is indicative of the presence of the L serovar in the sample, and wherein the absence of the amplification products is indicative of the absence of the L serovar in the sample,
wherein the at least one set of primers and the at least one detectable probe comprise:
a forward primer comprising a nucleic acid sequence selected from the group consisting of SEQ ID NOs: 1, 2, 6 and 7, or a combination thereof;
a reverse primer comprising a nucleic acid sequence selected from the group consisting of SEQ ID NOs: 3, 4, 8, and 9, or a combination thereof; and
a probe comprising a nucleic acid sequence selected from the group consisting of SEQ ID NOs: 5, 10 and 11, or a complement thereof.

2. The method of claim 1, wherein the hybridization step comprises contacting the amplification products with the detectable probe that is labelled with a donor fluorescent moiety and a corresponding acceptor moiety; and the detection step comprises detecting the presence or absence of fluorescence resonance energy transfer (FRET) between the donor fluorescent moiety and the acceptor moiety of the probes, wherein the presence or absence of fluorescence is indicative of the presence or absence of L serovar in the sample.

3. The method of claim 1, wherein the amplification step employs a polymerase enzyme having 5′ to 3′ nuclease activity.

4. The method of claim 1, wherein the sample is a biological sample.

5. The method of claim 4, wherein the biological sample is a vaginal swab specimen, a clinician-collected vaginal swab specimen, an endocervical swab specimen, an oropharyngeal (throat) swab specimen, or an anorectal swab specimen.

6. The method of claim 1, wherein the forward primer comprises a nucleic acid sequence selected from the group consisting of SEQ ID NOs: 1 and 2, or a combination thereof; the reverse primer comprises a nucleic acid sequence selected from the group consisting of SEQ ID NOs: 3 and 4, or a combination thereof; and the probe comprises a nucleic acid sequence of SEQ ID NO: 5, or a complement thereof.

7. The method of claim 1, wherein the forward primer comprises a nucleic acid sequence selected from the group consisting of SEQ ID NOs: 6 and 7, or a combination thereof; the reverse primer comprises a nucleic acid sequence selected from the group consisting of SEQ ID NOs: 8 and 9, or a combination thereof; and the probe comprises a nucleic acid sequence selected from the group consisting of SEQ ID NOs: 10 and 11, or a complement thereof.

8. The method of claim 1, wherein the forward primer comprises a nucleic acid sequence of SEQ ID NO: 7; the reverse primer comprises a nucleic acid sequence of SEQ ID NO: 9; and the probe comprises a nucleic acid sequence of SEQ ID NO: 11, or a complement thereof.

9. A method for detecting a lymphogranuloma venereum-causing serovar (L serovar) of Chlamydia trachomatis in a sample, the method comprising:
performing an amplification step comprising contacting the sample with at least one set of primers designed to target a 3′ end of the polymorphic membrane protein H (pmpH) gene of the L serovar to produce amplification products, if the L serovar is present in the sample;
performing a hybridization step, comprising contacting the amplification products, if the L serovar is present in the sample, with at least one detectable probe to target the pmpH gene of the L serovar; and
performing a detection step, comprising detecting the presence or absence of the amplification products, wherein the presence of the amplification products is indicative of the presence of the L serovar in the sample, and wherein the absence of the amplification products is indicative of the absence of the L serovar in the sample,
wherein the at least one set of primers is designed to target a region comprising at least a portion of the last 1000 nucleotides of the 3′ end of the pmpH gene of the L serovar.

10. The method of claim 9, wherein the at least one set of primers is designed to target a region comprising at least a portion of the last 500 nucleotides of the 3′ end of the pmpH gene of the L serovar.

11. The method of claim 9, wherein the at least one set of primers is designed to target a region comprising at least a portion of nucleotides 2600 to 2900 of the pmpH gene of the L1 serovar or a corresponding region of a different L serovar; or
wherein the at least one set of primers is designed to target a region consisting of at least a portion of nucleotides 2600 to 2800 of the pmpH gene of the L1 serovar or a corresponding region of a different L serovar.

12. A kit for detecting a pmpH gene of a lymphogranuloma venereum-causing serovar (L serovar) of Chlamydia trachomatis in a sample, the kit comprising amplification reagents comprising:
a DNA polymerase having 5′ to 3′ nuclease activity;
nucleotide monomers;
at least one pair of primers and at least one detectable probe comprising:
a forward primer comprising a nucleic acid sequence selected from the group consisting of SEQ ID NOs: 1, 2, 6 and 7 or any combination thereof;
a reverse primer comprising a nucleic acid sequence selected from the group consisting of SEQ ID NOs: 3, 4, 8 and 9, or a combination thereof; and
a detectable probe comprising a nucleic acid sequence selected from the group consisting of SEQ ID NOs: 5, 10 and 11, or a complement thereof.

13. The kit of claim 12, wherein the detectable probe is labeled with a donor fluorescent moiety and a corresponding acceptor moiety.

14. The kit of claim 12, wherein the forward primer comprises a nucleic acid sequence selected from the group consisting of SEQ ID NOs: 1 and 2, or a combination thereof; the reverse primer comprises a nucleic acid sequence selected from the group consisting of SEQ ID NOs: 3 and 4, or a combination thereof; and the probe comprises a nucleic acid sequence of SEQ ID NO: 5, or a complement thereof.

15. The kit of claim 12, wherein the forward primer comprises a nucleic acid sequence selected from the group consisting of SEQ ID NOs: 6 and 7, or a combination thereof; the reverse primer comprises a nucleic acid sequence selected from the group consisting of SEQ ID NOs: 8 and 9, or a combination thereof; and the probe comprises a nucleic acid sequence selected from the group consisting of SEQ ID NOs: 10 and 11, or a complement thereof.

16. The kit of claim 15, wherein the forward primer comprises a nucleic acid sequence of SEQ ID NO: 7; the reverse primer comprises a nucleic acid sequence of SEQ ID NO: 9; and the probe comprises a nucleic acid sequence of SEQ ID NO: 11, or a complement thereof.

17. (canceled)
